(19) United States
(12) Patent Application Publication
(10) Pub. No.: US 2008/0051647 A1
(43) Pub. Date: Feb. 28, 2008

(54) Non-invasive acquisition of large nerve action potentials (NAPs) with closely spaced surface electrodes and reduced stimulus artifacts

(76) Inventors: Changwang Wu, Newton, MA (US); Shai Gozani, Brookline, MA (US)

Correspondence Address: Mark J. Pandiscio;Pandiscio & Pandiscio, P.C., 470 Totten Pond Road, Waltham, MA, 02451-1914, US

(21) Appl. No.: 11/801,865

(22) Filed: May 11, 2007

Related U.S. Application Data
(60) Provisional application No. 60/799,512, filed on May 11, 2006.
(60) Provisional application No. 60/875,292, filed on Dec. 15, 2006.

(51) Int. Cl. A61N 1/04

(52) U.S. Cl. 600/382

(57) ABSTRACT

The present invention addresses the foregoing problems associated with the prior art by providing a novel method and apparatus for, non-invasively detecting large nerve action potentials (NAPs) while effectively minimizing or substantially eliminating stimulus artifacts, even where the stimulation site and the detection site are in close physical proximity to one another, e.g., within about 2 cm of one another.

### REFERENCE TO PENDING PRIOR PATENT APPLICATIONS

This patent application claims benefit of:

(i) pending prior U.S. Provisional Patent Application Ser. No. 60/799,512, filed May 11, 2006 by Changwang Wu et al. for NON-INVASIVE ACQUISITION OF GIANT NERVE ACTION POTENTIALS (Attorney's Docket No. NEURO-16 PROV); and

(ii) pending prior U.S. Provisional Patent Application Ser. No. 60/875,292, filed Dec. 15, 2006 by Michael Williams et al. for NEUROLOGICAL DIAGNOSTIC AND THERAPEUTIC SYSTEM WITH WIRELESS FUNCTIONAL MODULES (Attorney's Docket No. NEURO-22 PROV).

The two above-identified patent applications are hereby incorporated herein by reference.

### FIELD OF THE INVENTION

This invention relates to methods and apparatus for electrically stimulating a nerve and for detecting the evoked nerve action potentials (NAPs), for both diagnostic and therapeutic purposes.

### BACKGROUND OF THE INVENTION

U.S. Pat. Nos. 5,284,153 and 5,284,154 disclose a system for locating and identifying the function of specific peripheral nerves. The system of these patents generally comprises a surgical instrument for delivering an electrical stimulus to a nerve, a detector (i.e., a surface electrode) for detecting the electrical response of the nerve to the stimulus delivered by the surgical instrument (i.e., a nerve action potential, also known as an NAP), a recorder for recording the intensity of the electrical response of the nerve, and means for evaluating the recorded intensity of the electrical response of the nerve against predetermined criteria, whereby to determine the proximity of the surgical instrument to the nerve. Among other things, the system can be used with sensory nerves, in which case the detected nerve action potential (NAP) may be referred to as a sensory nerve action potential (SNAP).

One problem with the system of the aforementioned U.S. Pat. Nos. 5,284,153 and 5,284,154 is that the system is susceptible to contamination by stimulus artifacts. More particularly, the system of the aforementioned U.S. Pat. Nos. 5,284,153 and 5,284,154 operates by (i) applying an electrical stimulation pulse at a stimulation site, and (ii) detecting the evoked nerve action potential (NAP) at the detection site. If the detector picks up an artifact of the electrical stimulation pulse (i.e., a stimulus artifact) simultaneously with the evoked nerve action potential (NAP), and if the intensity of the stimulus artifact is significant vis-à-vis the intensity of the nerve action potential (NAP), the integrity of the detected signal (sometimes referred to as “the trace”) is necessarily diminished and the usefulness of the detected signal may be significantly reduced.

Unfortunately, with the system of the aforementioned U.S. Pat. Nos. 5,284,153 and 5,284,154, the detector comprises one or more surface electrodes. While these surface electrodes are non-invasive and highly convenient to use, the surface electrodes also yield a relatively low nerve signal (i.e., a NAP of relatively low intensity) if they are not placed close enough to the stimulation site. By way of example but not limitation, the amplitude (i.e., intensity) of a nerve action potential for the median nerve is typically no more than about 110 uV. Furthermore, the amplitude of the nerve action potentials (NAPs) at the detection site can be even further reduced due to pathological reasons, e.g., if the nerve extending between the stimulation site and the detection site has conduction problems, and/or if the nerve is damaged, and/or if the conduction velocity of the individual nerve fibers vary (which can cause phase cancellation) such as with segmental demyelination, etc.

Thus, in applications such as, but not limited to, locating specific peripheral nerves (e.g., the median nerve), it is preferable to place the detector's surface electrodes close to the stimulation site, in order to obtain reliable, high intensity nerve action potentials (NAPs) evoked by the electrical stimulus.

However, if the detector's surface electrodes are placed close to the stimulation site so as to yield a higher intensity nerve action potential (NAP), the stimulus artifacts can be substantial relative to the nerve signal itself. Specifically, in this situation, the stimulus artifacts will typically be manifested as relatively large displacements of (i) the baseline of the nerve signal, and (ii) the nerve signal itself. These large stimulus artifact displacements can interfere with the relatively modest amplitudes of the nerve action potentials (NAPs) obtained by the detector's surface electrodes thereby undermining the usefulness of the detected signal (i.e., the trace).

Thus, with the system of the aforementioned U.S. Pat. Nos. 5,284,153 and 5,284,154, in order to avoid stimulus artifact contamination of the detected nerve action potential (NAP), the detecting surface electrodes must generally be placed an adequate distance from the stimulation site, in order to adequately reduce the magnitude of the stimulus artifacts vis-à-vis the NAPs. This may not always be possible or convenient, depending upon the specific nerve which is being studied and/or on variations in patient anatomy, etc.

As a result, several approaches have been developed to minimize or substantially eliminate the aforementioned stimulus artifacts.

One simple and effective approach for eliminating stimulus artifacts involves biphasic stimulation. More particularly, with this approach, a positive pulse (i.e., a current flowing from anode to cathode, which stimulates the nerve located under the cathode) is first applied to the tissue, and then a negative pulse (i.e., a current flowing from cathode to anode, which will not stimulate the nerve located under the cathode because the negative pulse is delivered when the nerve is refractory due to the stimulation of the positive pulse) is applied to the tissue, with the amplitude of the negative pulse being adjusted so as to cancel any stimulus artifact created by the positive pulse. Furthermore, with such a biphasic stimulation approach, it has been found that the results can be further improved by configuring the detector so that its recording amplifier uses a high pass filter which has a relatively low cut-off frequency.

However, even using biphasic stimulation with a recording amplifier having a high pass filter with a relatively low cut-off frequency does not eliminate stimulus artifacts when the separation distance between the stimulation site and the detection site is small. In particular, it has been found that in many situations, a separation distance of approximately 5.5 cm is still required between the stimulation site and the detection site in order to sufficiently minimize stimulus artifacts when using surface electrodes for the detector. Such a separation distance may still be too large for many applications.

Furthermore, acquiring larger nerve action potentials (NAPs) is desired in many applications in order to increase the signal-to-noise ratio of the nerve signal. One preferred way to acquire larger nerve action potentials (NAPs) is to replace the detector's surface electrodes with needle electrodes. More particularly, this approach uses a bipolar needle electrode (or a pair of monopolar needle electrodes) as the detecting electrodes, with the bipolar needle electrode (or monopolar needle electrodes) penetrating the skin and being positioned near the nerve. However, this approach is generally not preferred, since it is a highly invasive approach.

Therefore, the need exists for a new system which can, non-invasively, acquire large nerve action potentials (NAPs) while effectively minimizing or substantially eliminating stimulus artifacts, even where the stimulation site and the detection site are in close physical proximity to one another, e.g., within about 2 cm of one another.

### SUMMARY OF THE INVENTION

The present invention addresses the foregoing problems associated with the prior art by providing a novel method and apparatus for, non-invasively detecting large nerve action potentials (NAPs) while effectively minimizing or substantially eliminating stimulus artifacts, even where the stimulation site and the detection site are in close physical proximity to one another, e.g., within about 2 cm of one another.

More particularly, the novel apparatus of the present invention comprises a stimulator and a detector. The stimulator applies an electrical stimulus to a nerve at a stimulation site, and the detector detects the evoked nerve action potential (NAP) at a detection site. The novel apparatus of the present invention is capable of detecting the voltage between the anode and the cathode, hereafter called Residual Voltage, or RV. The means for detecting the RV could be part of the stimulator, or a separate module.

The stimulator is configured to provide biphasic stimulation to the tissue, i.e., the stimulator first delivers a positive pulse (i.e., a current flowing from anode to cathode) to the tissue, and then the stimulator delivers a negative pulse (i.e., a current flowing from cathode to anode) to the tissue so as to cancel any stimulus artifact created by the positive pulse. Significantly, with the present invention, the time duration of the negative pulse is adjusted, while keeping the amplitude of the negative pulse constant, so as to minimize or substantially eliminate the stimulus artifact. This novel approach is in marked contrast to the prior art, which adjusts the amplitude of the negative pulse so as to cancel any stimulus artifact created by the positive pulse.

Due to the novel approach of the present invention, stimulus artifacts can be minimized or substantially eliminated, either (i) by utilizing a feedback mechanism applied across multiple stimulations or (ii) in real-time, even where the detector comprises surface electrodes and those surface electrodes are located quite close to the stimulation site, e.g., as close as about 2 cm.

In further accordance with the present invention, the time duration of the negative pulse can be manually or automatically adjusted so as to minimize the stimulus artifact. The stimulator may also short the anode and cathode so as to speed up the rate at which the patient's tissue discharges any acquired electric charge at the stimulation site, which can also serve to reduce stimulus artifacts.

The detector comprises at least one surface electrode and an analog front end (AFE). The AFE in turn comprises an instrumentation amplifier (INA), a filter and main amplifiers. The INA is configured to detect signals that have high source impedance. The detector's detecting electrodes and the AFE detect the trace signal, which is then sent to a controller/monitor for recording, measuring and analyzing. Among other things, the AFE has broad bandwidth. The low cut-off frequency of the AFE is very low, e.g., about 0.3 Hz. The high cut-off frequency of the AFE is relatively high, e.g., above about 20 KHz. The INA preferably has a comparably broad dynamic range.

The present invention can be used for both diagnostic and therapeutic purposes.

In one preferred form of the present invention, there is provided apparatus for acquiring a nerve action potential (NAP) from a patient, the apparatus comprising:

a stimulator and a pair of stimulator electrodes connected to the stimulator for applying an electrical stimulus to the patient so as to evoke a nerve action potential (NAP) in the patient;

a detector and a pair of detector electrodes connected to the detector for acquiring a trace signal from the patient, wherein the trace signal includes the nerve action potential (NAP); and

shorting apparatus for shorting the pair of stimulator electrodes after application of the electrical stimulus to the patient in order to minimize the presence of stimulus artifacts in the trace signal.

In another preferred form of the present invention, there is provided a method for acquiring a nerve action potential (NAP) from a patient, the method comprising the steps of:

applying an electrical stimulus to the patient using a stimulator and a pair of stimulator electrodes connected to the stimulator so as to evoke a nerve action potential (NAP) in the patient; and

acquiring a trace signal from the patient which includes the nerve action potential (NAP);

wherein the pair of stimulator electrodes are shorted after the electrical stimulus has been applied to the patient in order to minimize the presence of stimulus artifacts in the trace signal.

In another preferred form of the present invention, there is provided apparatus for acquiring a nerve action potential (NAP) from a patient, the apparatus comprising:

a stimulator and a pair of stimulator electrodes connected to the stimulator for applying an electrical stimulus to the patient so as to evoke a nerve action potential (NAP) in the patient;

a detector and a pair of detector electrodes connected to the detector for acquiring a trace signal from the patient, wherein the trace signal includes the nerve action potential (NAP);

wherein the stimulator is configured to produce a biphasic electrical stimulus consisting of a positive pulse followed by a negative pulse;

and further wherein the stimulator is configured to minimize the presence of stimulus artifacts in the trace signal by regulating the time duration of the negative pulse.

In another preferred form of the present invention, there is provided a method for acquiring a nerve action potential (NAP) from a patient, the method comprising the steps of:

applying an electrical stimulus to the patient so as to evoke a nerve action potential (NAP) in the patient, wherein the electrical stimulus comprises a biphasic electrical stimulus comprising a positive pulse followed by a negative pulse; and

acquiring a trace signal from the patient which includes the nerve action potential (NAP);

wherein the time duration of the negative pulse is regulated so as to minimize the presence of stimulus artifacts in the trace signal.

In another preferred form of the present invention, there is provided apparatus for acquiring a nerve action potential (NAP) from a patient, the apparatus comprising:

a stimulator and a pair of stimulator electrodes connected to the stimulator for applying an electrical stimulus to the patient so as to evoke a nerve action potential (NAP) in the patient, wherein the stimulator is configured to produce a biphasic electrical stimulus consisting of a positive pulse followed by a negative pulse;

a detector and a pair of detector electrodes connected to the detector for acquiring a trace signal from the patient, wherein the trace signal includes the nerve action potential; and

a determining component for determining the amplitude of a stimulus artifact present in the trace signal;

wherein the stimulator is configured to minimize the presence of stimulus artifacts in the trace signal by regulating the time duration of the negative pulse based on the amplitude of a stimulus artifact present in a prior trace signal as determined by the determining component.

In another preferred form of the present invention, there is provided a method for acquiring a nerve action potential (NAP) from a patient, the method comprising the steps of:

applying an electrical stimulus to the patient so as to evoke a nerve action potential (NAP) in the patient, wherein the electrical stimulus comprises a biphasic electrical stimulus comprising of a positive pulse followed by a negative pulse;

acquiring a trace signal from the patient which includes the nerve action potential (NAP);

determining the amplitude of a stimulus artifact present in the trace signal; and

regulating the time duration of the negative pulse in a subsequent biphasic electrical stimulus so as to minimize the presence of stimulus artifacts in a current trace signal based on the amplitude of a stimulus artifact present in a prior trace signal.

In another preferred form of the present invention, there is provided apparatus for measuring the stimulus artifact present when acquiring a nerve action potential (NAP) from a patient, the apparatus comprising:

a stimulator and a pair of stimulator electrodes connected to the stimulator for applying an electrical stimulus to the patient so as to evoke a nerve action potential (NAP) in the patient;

a detector and a pair of detector electrodes connected to the detector for acquiring a trace signal from the patient, wherein the trace signal includes the nerve action potential (NAP); and

a measuring component for measuring the voltage present between the pair of stimulator electrodes after application of the electrical stimulus to the patient.

In another preferred form of the present invention, there is provided a method for measuring the stimulus artifact present when acquiring a nerve action potential (NAP) from a patient, the method comprising the steps of:

applying an electrical stimulus to the patient using a pair of stimulator electrodes, so as to evoke a nerve action potential (NAP) in the patient; and

acquiring a trace signal from the patient which includes the nerve action potential (NAP);

wherein the voltage between the pair of stimulator electrodes is measured after the beginning of application of the electrical stimulus to the patient.

In another preferred form of the present invention, there is provided apparatus for acquiring a large nerve action potential (NAP) from a patient, the apparatus comprising:

a stimulator and a pair of stimulator electrodes connected to the stimulator for applying an electrical stimulus to the patient so as to evoke a nerve action potential (NAP) in the patient; and

a detector and a pair of detector electrodes connected to the detector for acquiring a trace signal from a patient, wherein the trace signal includes the nerve action potential (NAP);

wherein at least one of the pair of detector electrodes is a surface electrode and is positioned less than 3 cm from the stimulator electrodes.

In another preferred form of the present invention, there is provided a method for acquiring large nerve action potential (NAP) from a patient, the method comprising the steps of:

applying an electrical stimulus to the patient using a pair of stimulator electrodes so as to evoke a nerve action potential (NAP) in the patient; and

acquiring a trace signal from the patient which includes the nerve action potential (NAP), wherein the trace signal is acquired from the patient using a pair of detector electrodes;

wherein at least one of the pair of the detector electrodes is a surface electrode and is placed less than 3 cm from the stimulator electrodes.

### BRIEF DESCRIPTION OF THE DRAWINGS

These and other objects and features of the present invention will be more fully disclosed or rendered obvious by the following detailed description of the preferred embodiments of the invention, which is to be read in conjunction with the accompanying drawings wherein like numbers refer to like elements, and further where:

FIG. 1 is a schematic block diagram of the preferred system of the present invention;

FIG. 2 is a schematic diagram showing an impedance model of the patient's tissue;

FIG. 3 is a schematic illustration showing the detected nerve action potential (NAP) contaminated by a stimulus artifact;

FIG. 4 is a schematic illustration showing the detected nerve action potential (NAP) without contamination by a stimulus artifact;

FIG. 5 is a schematic illustration showing the distance-NAP amplitude relationship with a superficial peroneal nerve test;

FIG. 6 is a schematic illustration showing a typical electrode configuration for a median nerve test using surface electrodes;

FIG. 7 is a schematic illustration showing the nerve action potential (NAP) detected in a median nerve test using surface electrodes;

FIG. 8 is a schematic illustration showing the electrode configuration for a median nerve test with needle stimulation;

FIG. 9 is a schematic illustration showing the nerve action potential (NAP) detected in the median nerve test using a needle as the stimulator cathode;

FIGS. 10-12 are schematic illustrations showing the relationship between the stimulus artifact and the voltage present between the stimulator's anode and cathode; and

FIG. 13 is a schematic illustration showing the current and voltage waveforms between the stimulator's anode and cathode.

### DETAILED DESCRIPTION OF THE PREFERRED EMBODIMENTS

### Reducing Stimulus Artifacts by Shorting the Stimulator's Anode and Cathode

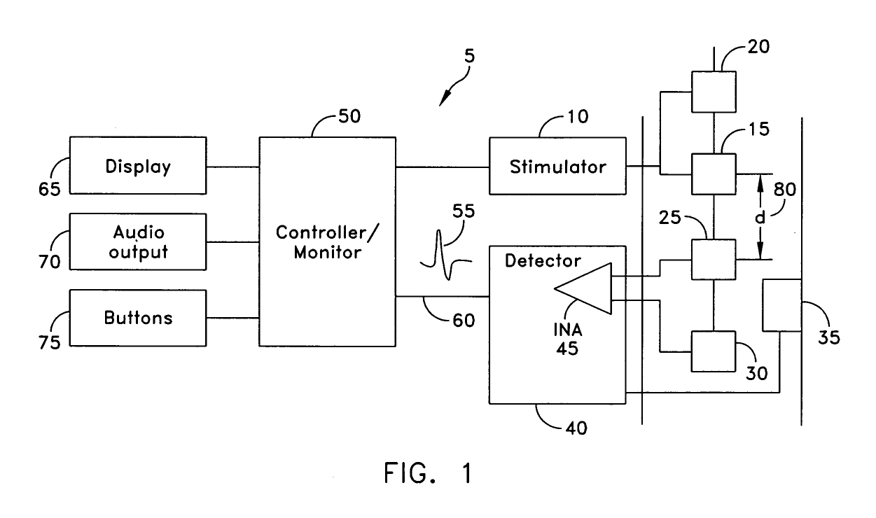

Looking first at FIG. 1, there is shown a device 5 which comprises a preferred embodiment of the present invention. More particularly, device 5 comprises apparatus for, non-invasively detecting large nerve action potentials (NAPs) while effectively minimizing or substantially eliminating stimulus artifacts, even where the stimulation site and the detection site are in close physical proximity to one another, e.g., within about 2 cm of one another.

NAP acquisition device 5 comprises a constant current stimulator circuit (also known as the stimulator) 10 that delivers an electrical stimulus to the stimulating electrodes 15 and 20 so as to stimulate a nerve of a patient. The evoked nerve action potential (NAP) is detected by a pair of surface electrodes 25 and 30, preferably in conjunction with a reference surface electrode 35. Electrodes 25 and 30 (and preferably also 35) are connected to a detector circuit (also known as the detector) 40 which includes an INA 45. A controller/monitor 50 operates stimulator 10 and receives the output trace 55 from detector 40. The controller/monitor 50 may also receive the Residual Voltage trace from stimulator 10. The connection 60 between detector 40 and controller/monitor 50 may be wired or wireless. Display 65, audio output 70 and buttons 75, as well as other optional input/output controls, permit a user to interact with NAP acquisition device 5. Further details of the construction and operation of NAP acquisition device 5 will hereinafter be provided.

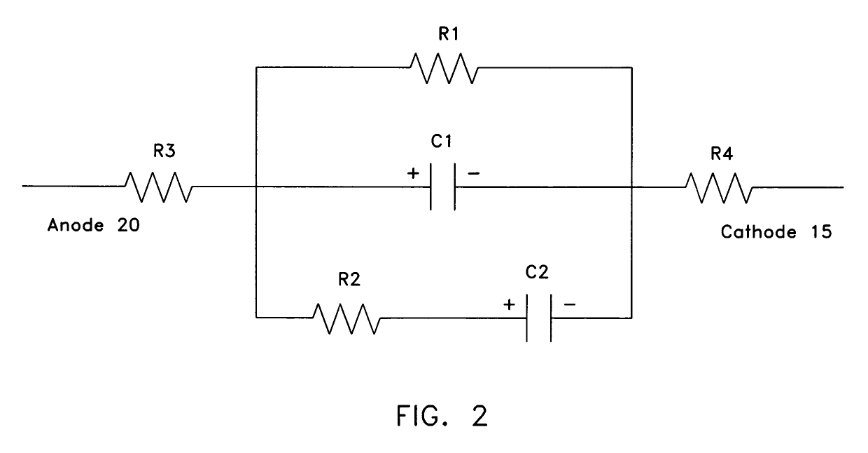

Significantly, the distance d between the stimulation site and the detection site (shown in FIG. 2 at 80) may be quite short, e.g., approximately 2 cm.

FIG. 2 is a simplified impedance model of the patient's tissue. The positive current pulse from stimulator circuit 10 flows from anode electrode 20 to cathode electrode 15, which charges capacitors C1 and C2. When the positive current pulse from stimulator circuit 10 stops, capacitors C1 and C2 discharge. For the same amount of stimulation current, the speed of discharge depends on the values of resistors R1 and R2 and capacitors C1 and C2, which vary (i) from subject to subject, (ii) for the same subject, from tissue condition to tissue condition, and (iii) for the same subject, from electrode configuration to electrode configuration. Thus it will be seen from the simplified impedance model of FIG. 2 that the patient's tissue will first be charged by the positive pulse from stimulator circuit 10, and then the patient's tissue will discharge the acquired electric charge, i.e., in a “tissue discharge current”.

Looking again now at FIG. 1, the detecting electrodes 25 and 30, and the detector circuit 40, detect the nerve action potential (NAP) evoked by the positive pulse delivered by stimulator 10 and stimulator electrodes 15 and 20. The connection 60 between detector circuit 40 and controller/monitor 50 may be wired or wireless. By way of example but not limitation, the connection 60 between detector circuit 40 and controller/monitor 50 may be a wireless connection of the sort disclosed in pending prior U.S. Provisional Patent Application Ser. No. 60/875,292, filed Dec. 15, 2006 by Michael Williams et al. for NEUROLOGICAL DIAGNOSTIC AND THERAPEUTIC SYSTEM WITH WIRELESS FUNCTIONAL MODULES (Attorney's Docket No. NEURO-22 PROV), which patent application is hereby incorporated herein by reference.

In addition to detecting the nerve action potential (NAP) evoked by the positive pulse, the detecting electrodes 25 and 30 will also detect the positive pulse delivered by stimulator circuit 10 and stimulator electrodes 15 and 20 and, more significantly, the detecting electrodes will also detect the tissue discharge current as the energy accumulated in the tissue during stimulation is discharged, which is the major source of stimulus artifact. As noted above, when the conduction distance d, identified at 80, is short, the stimulus artifact is greater because the detection site is closer to the stimulation site, and therefore more of the accumulated electric discharge in the tissue reaches the detector's electrodes. This is essentially why prior art monophasic devices (i.e., those devices using a monophasic, not biphasic, waveform) required substantial stimulator/detector separation in order to minimize the stimulus artifact.

In order to detect the nerve action potential (NAP) without stimulus artifacts, the tissue discharge current must be very low (if not zero) and stable by the time the nerve action potential (NAP) arrives to the detecting electrode 25. If, when viewed in the context of the tissue impedance model of FIG. 2, the values of C1 and/or C2 are not very small, and the values of R1 and/or R2 are large, the self-discharge (i.e., the tissue discharge) will be relatively slow and will contaminate the detected nerve action potential (NAP) because of the larger RC time constants.

In order to speed up the tissue discharge process, the stimulator circuit 10 of the present invention may short the anode 20 and cathode 15 if desired. Again, in the context of the tissue impedance model of FIG. 2, when the anode 20 and cathode 15 are shorted in this way, if the values of R3 and R4 are small, capacitor C1 can be quickly discharged. If the value of R2 is also small, capacitor C2 will also be quickly discharged. Thus, shorting the anode 20 and the cathode 15 can speed up the discharge of the acquired electric charge in the tissue.

Shorting the anode and cathode helps to speed up the discharge of residual energy stored in capacitor C1 and, to a lesser degree, in capacitor C2 (i.e., it can help speed up the discharge of the acquired electric current in the tissue, and hence reduce the stimulus artifact). However, shorting the anode and cathode also has the following disadvantages:

(1) it may generate a high current spike—the peak of the tissue discharge current is determined by the residual voltage at capacitor C1 divided by the value of R3 and R4, and this high current spike may not be safe for the patient, particularly when using needle stimulation; and

(2) when the value of R2 is large, capacitor C2 cannot be quickly discharged.

### Biphasic Stimulation

As noted above, biphasic stimulation can also be used to reduce a stimulus artifact.

In the prior art, the time duration of the negative pulse is the same as the time duration of the positive pulse. The amplitude of the negative pulse is adjusted so as to minimize the stimulus artifact. Thus, when the stimulus artifact is “falling down” (i.e., introducing a drop in the amplitude of the trace signal, signifying that the tissue is under-discharged), the amplitude of the negative pulse of the next stimulus will be increased. However, the negative pulse can itself introduce an artifact, with opposite direction, when the amplitude of the negative pulse is set too high (i.e., introducing a rise in the amplitude of the trace signal, signifying that the tissue is over-discharged). With the prior art approach, multiple voltage levels may need to be tried for the negative pulse before the optimum amplitude for the negative pulse is determined in order to minimize the stimulus artifact. This can be inconvenient for the user.

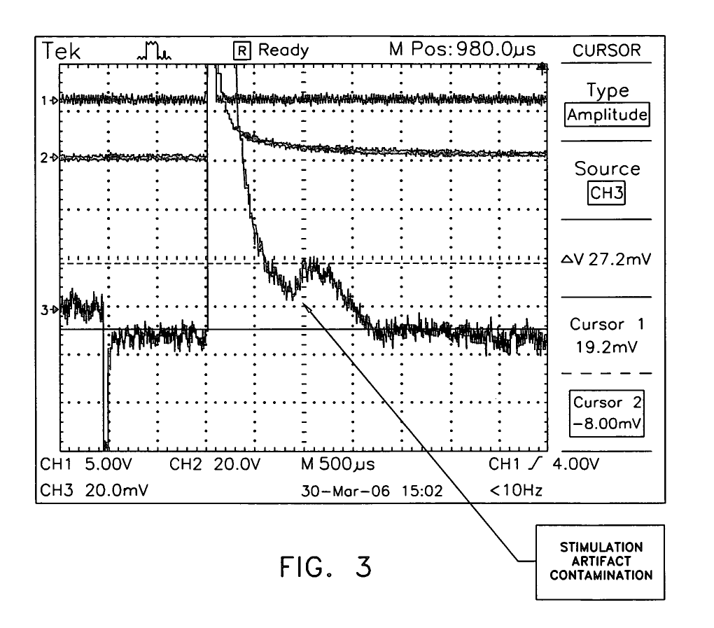

The novel device of the present invention also uses biphasic stimulation to minimize or substantially eliminate stimulus artifacts for stimulation studies using a surface electrode or needle as the cathode. However, and significantly, the present device is configured to adjust the time duration of the negative pulse, not the amplitude of the negative pulse, in order to minimize or substantially eliminate the stimulus artifact. In this way, the negative pulse can be terminated at any time when the stimulus artifact (if it is monitored) is within an acceptable limit, thereby avoiding over-discharge without having to try multiple voltage levels or time durations for the negative pulse. Generally, only the stimulus artifact in the time period of the nerve action potential (NAP) signal should be minimized. See FIGS. 3, 4 and 9-12. The up-peak of the nerve action potential (NAP) is the positive peak of the nerve action potential (NAP) signal. The down-peak of the nerve action potential (NAP) is the negative peak of the nerve action potential (NAP) signal. The up-peak arrives before the down-peak. If the up-peak amplitude of the nerve action potential (NAP) is to be measured, then the presence of stimulus artifacts at the time of the up-peak should be minimized. If the down-peak amplitude of the nerve action potential (NAP) is to be measured, then the presence of stimulus artifacts at the time of the down-peak should be minimized. If the peak-to-peak amplitude of the nerve action potential (NAP) is to be measured, then the presence of stimulus artifacts at both the time of up-peak and the time of down-peak should be minimized. It is possible that when the presence of stimulus artifacts at the time of the up-peak is minimized, then the presence of stimulus artifacts at the time of the down-peak would have been be minimized. If the characteristics of the whole nerve action potential (NAP) signal are to be measured, e.g., the latency and the duration of the nerve action potential (NAP) signal, then it is preferred to minimize the presence of stimulus artifacts in the overall trace signal after the end of stimulus.

This novel approach is in marked contrast to the approach of prior art biphasic stimulators, which rely on an adjustment of the amplitude of the negative pulse to minimize the stimulus artifact, with the attendant disadvantages of inconvenience, delay and inaccuracy noted above.

Thus, with the present invention, and looking again at FIG. 1, stimulator circuit 10 is configured to deliver biphasic stimulation, i.e., to first deliver a positive pulse (i.e., a current flowing from anode 20 to cathode 15), and then deliver a negative pulse (i.e., a current flowing from cathode 15 to anode 20). Also in accordance with the present invention, stimulator circuit 10 is configured to adjust the time duration of the negative pulse, while keeping the amplitude of the negative pulse constant, so as to minimize stimulus artifacts.

Furthermore, for surface electrode stimulation studies, the present invention is preferably also configured so as to internally short the anode 20 and cathode 15 for a short period of time before the negative pulse is delivered. This approach further reduces the time for eliminating a stimulus artifact by, when considered in the context of the tissue model of FIG. 2, depleting the residual energy stored in capacitors C1 and C2.

As noted above, detector 40 comprises an analog front end (AFE) which in turn comprises an instrumentation amplifier (INA) 45, a filter and main amplifiers. The AFE of detector 40 comprises a high pass filter and a low pass filter, with the filters being configured so as to provide a relatively broad bandwidth. More particularly, in order to reduce stimulus artifact, the cut-off frequency of the high pass filter should be low enough that the trailing edge of any offset will change slowly enough that there is no interference with the evoked nerve signal. At the same time, the AFE of the detector has a low pass filter which has a high cut-off frequency. For a 100 us positive pulse, the time duration of the optimum negative pulse that eliminates the stimulation artifact to the minimum level will be less than 100 us. If the cut-off frequency of the low pass filter is too low, e.g., 3 KHz, the passage of the positive pulse and the negative pulse will introduce an exponential tail into the nerve signal that arrives at the detecting electrodes shortly (e.g., 200 us) after stimulation occurs. A wider bandwidth will have no exponential tail artifact because of the fast response time provided by the wide bandwidth.

The detector circuit 40 has the following characteristics: the output voltage range of the INA 45 is about −5V to about +5V. In order to prevent the INA 45 from saturating, the gain of the INA should be small when the amplitude of the positive pulse and the negative pulse is high. The AFE of detector 40 can be designed to have a broader output voltage range, e.g., approximately +/−15V, so as to avoid any saturation problems.

FIG. 3 shows a superficial peroneal nerve action potential (NAP) evoked by a conventional monophasic, constant-current electrical stimulus using a surface tab electrode as the cathode. The conduction distance d is 3.8 cm from the center of stimulating cathode 15 to the center of detecting electrode 25. The stimulation current is 20 mA. The gain of the AFE is 493. The bandwidth of the AFE is about 0.32 Hz to about 31 KHz. As would be expected, the SNAP in FIG. 3 is contaminated by a stimulus artifact.

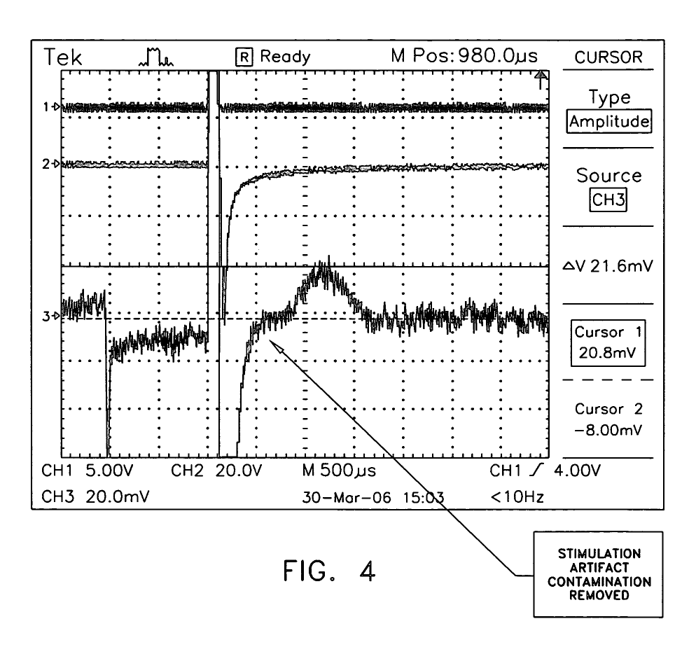

FIG. 4 shows a superficial peroneal nerve action potential (NAP) evoked by a preferred embodiment of the present invention, i.e., a novel biphasic, constant-current stimulation using a surface tab electrode as the cathode. The conduction distance d is 3.8 cm from the center of stimulating cathode 15 to the center of detecting electrode 25. The stimulation current is 20 mA. The gain of the AFE is 493. The bandwidth of the AFE is about 0.32 Hz to about 31 KHz. In accordance with the present invention, the SNAP in FIG. 4 is not contaminated by a stimulus artifact.

The stimulus artifact present in FIG. 3 and missing from FIG. 4 was removed by utilizing the approach of the present invention. More specifically, the SNAP was induced by stimulating the tissue with a biphasic signal, i.e., by first delivering a positive pulse (flowing from anode to cathode), and then delivering a negative pulse (flowing from cathode to anode). In accordance with the present invention, stimulator circuit 10 is configured to adjust the time duration of the negative pulse, while keeping the amplitude of the negative pulse constant, so as to minimize the stimulus artifact.

The artifact elimination method described above allows users to place the detection electrodes close to the stimulation site and still detect a true NAP without a superimposed stimulus artifact contaminating the nerve signal. This is a significant advance over the prior art.

### Relationship Between NAP Amplitude and the Conduction Distance D

During the development of this invention, the relationship between NAP amplitude and the conduction distance d was also clearly established: for the same stimulation current intensity and the same stimulation site, when d is decreased, the amplitude of the NAP is increased, and when d is increased, the amplitude of the NAP is decreased.

This amplitude-distance relationship was validated by using a superficial peroneal nerve and a sural nerve.

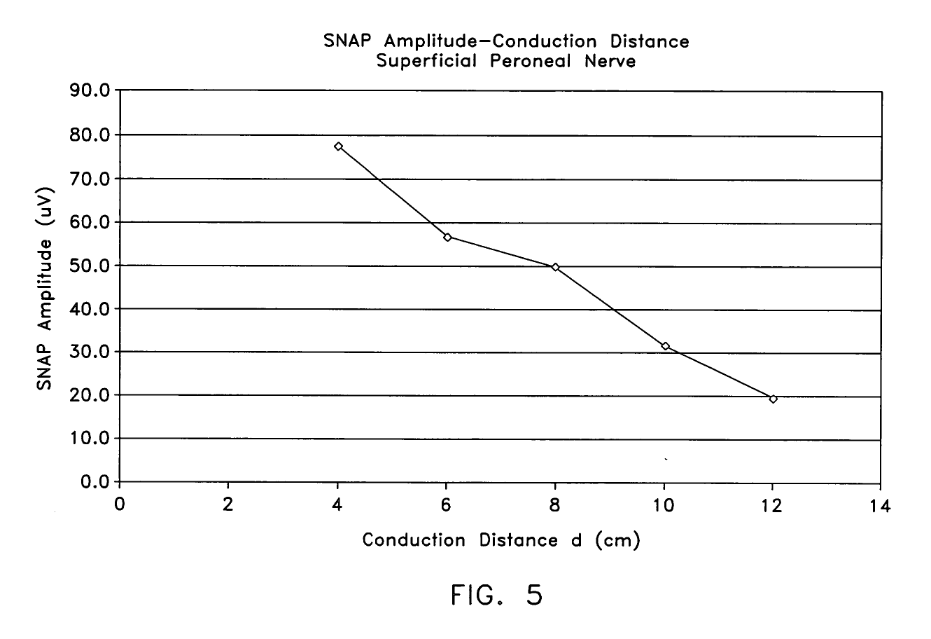

FIG. 5 shows the test results for a superficial peroneal nerve. The patient was a healthy 40 year old male. The stimulation current was 15 mA for 0.1 ms duration. The cathode was fixed in position 16 cm above the ankle. The detecting electrodes were moved, in steps, toward the cathode from a distal position. When the conduction distance d was 4 cm, the SNAP amplitude was 77.1 uV. When the conduction distance d was 11.8 cm, the SNAP amplitude was 19.5 uV. In another test, the cathode was fixed at the ankle, and the detecting electrodes were moved, in steps, toward the cathode from a proximal position. In yet another test, the detecting electrodes were fixed at the ankle, and the stimulation electrodes were moved toward the detecting electrodes from a proximal position. All three tests yielded consistent results: when the conduction distance d was decreased, the amplitude of the SNAP was increased, and when the conduction distance d was increased, the amplitude of the SNAP was decreased.

Similar tests were performed with a sural nerve, and the results were consistent with the foregoing.

When the conduction distance d was as short as 2 cm to 3 cm for the median nerve test, the present invention detected very large nerve action potentials (NAPs). Again, this is a significant improvement over the prior art.

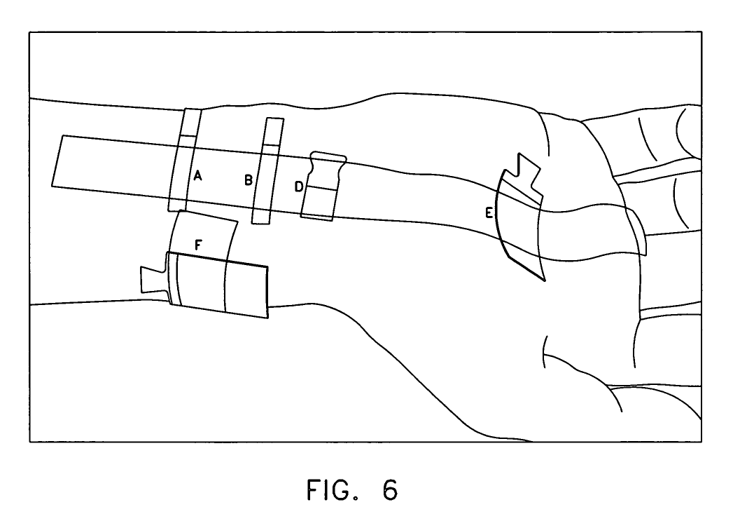

FIG. 6 shows an example of electrode configurations for a median nerve test using a surface electrode as the cathode. The electrodes were connected to the stimulator and the AFE of the detector as follows:

| | | | | |
|---|---|---|---|---|
| Cathode: D | Anode: E | AFE−: B | AFE+: A | Ref: F |

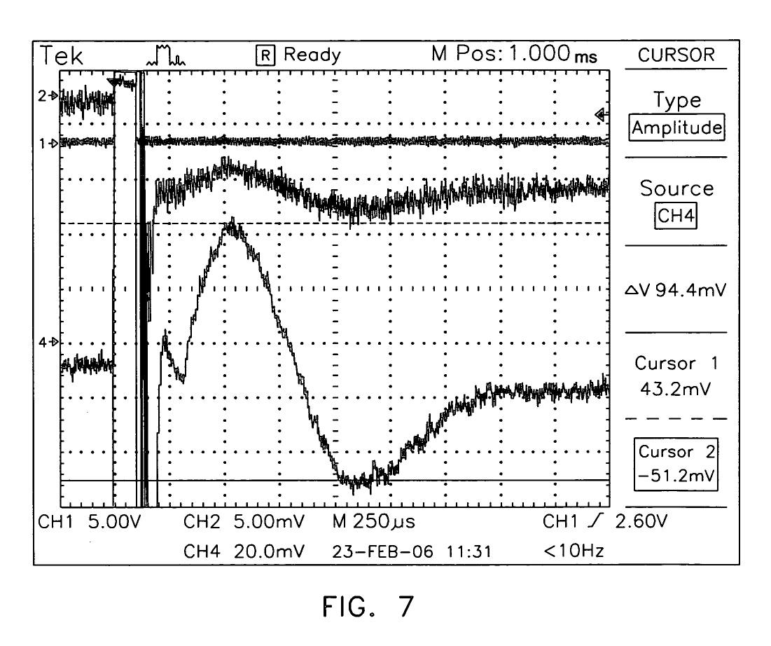

FIG. 7 shows the waveform recorded by the oscilloscope using the foregoing arrangement. There are 3 traces on the drawing, marked (on the left of the diagram) as 1, 2 and 4, corresponding to channels 1, 2 and 4 of the oscilloscope (channel 3 was not used). Channel 1 is the trigger signal. Channel 2 is the INA output with gain of 10.78. Channel 4 is the AFE output with gain of 250.7. The bandwidth of the INA is 0-500 KHz. The bandwidth of the AFE is 0-25.8 KHz. The stimulation current is a 100 us, 30 mA positive pulse, followed by discharge with anode and cathode shorted, and then followed by 10 us 30 mA negative pulse. The peak-to-peak amplitude of the signal output from the AFE is 94.4 mV, which is equivalent to 94.4 mV/250.7=377 uV input NAP signal.

The same test protocol was carried out on the median nerve of two additional patients. The supramaximal amplitudes of NAP signals were measured at 351 uV (at 21 mA) and 380 uV (at 20 mA), respectively. Compared to the reported prior art amplitude of approximately 110 uV, these signals are 3 to 4 times larger than those achieved using prior art techniques. Again, this is a significant improvement over the prior art.

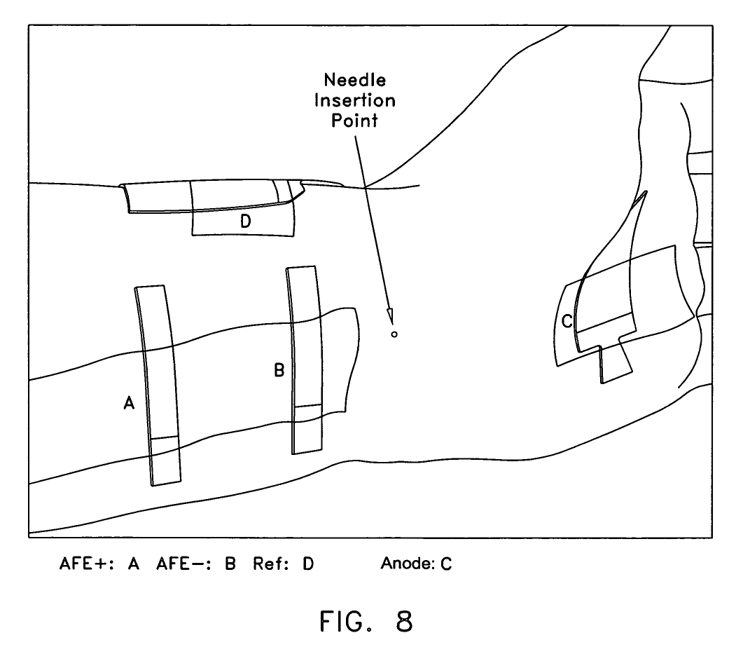

A similar approach can be used with needle stimulation. FIG. 8 shows an example of electrode configurations for a median nerve test using a needle as the cathode. In this test, the surface electrode cathode was replaced with a needle (TECA 902-DMG50, length 50 mm, diameter 0.46 mm (26 gauge), recording area 0.34 mm²). The cathode needle insertion point was 2 cm from the center of “B”. The needle was inserted toward the wrist for about 4 mm to 5 mm.

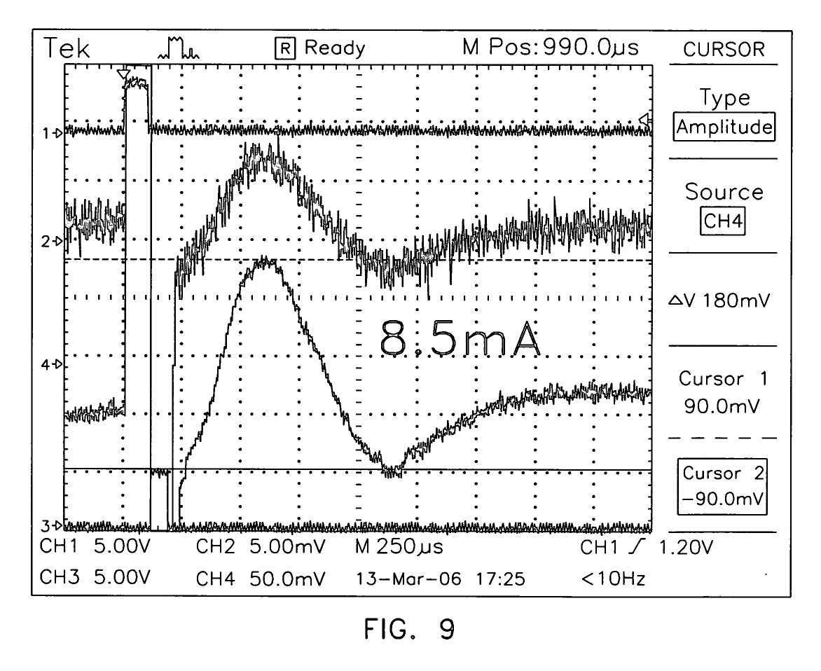

FIG. 9 is the waveform recorded by the oscilloscope using the foregoing arrangement. There are 4 traces on the drawing, marked (on the left of the diagram) as 1, 2, 3 and 4, corresponding to channel numbers 1, 2, 3 and 4 of the oscilloscope. Channel 1 is the trigger signal. Channel 2 is the INA output with gain of 26. Channel 4 is the AFE output with gain of 501. The bandwidth of the INA is 0-500 KHz. The bandwidth of the AFE is 0.32-31 KHz. Channel 3 is the negative pulse control signal. The stimulation current is a 100 us, 8.5 mA positive pulse, followed by a 70 us, 8.5 mA negative pulse. To avoid a potentially unsafe high discharge spike current flowing between the anode and cathode, the anode and cathode were not shorted. The peak-to-peak amplitude of the signal output from the AFE is 180 mV, which is equivalent to 180 mV/501=359 uV input NAP signal. Compared to the reported prior art amplitude of approximately 110 uV, these signals are 3 to 4 times larger than those achieved using prior art techniques. Again, this is a significant improvement over the prior art.

### Automatic Stimulus Artifact Removal

In another significant aspect of the present invention, the stimulus artifact can be minimized or substantially eliminated automatically. This can be done either (i) by utilizing a feedback mechanism applied across multiple stimulations, or (ii) in real-time. Each of these two approaches will now be separately discussed.

### (i) Automatic Stimulus Artifact Removal

### by Utilizing a Feedback Mechanism Applied Across Multiple Stimulations

For situations which allow multiple tries to find the optimum time duration of the negative pulse of the biphasic stimulation, the stimulus artifact can be removed by (a) using the detected trace signal output from detector 40, or (b) using the detected voltage signal between anode 20 and cathode 15.

### (a) Using The Detected Trace Signal Output From Detector 40

The controller/monitor 50 measures and analyzes the detected trace signal output from detector 40, determines the stimulus artifact, and then compares the amplitude of the stimulus artifact contaminating the detected NAP to a pre-defined limit. When the stimulus artifact is outside that pre-defined limit and is falling downward (signifying that the tissue is under-discharged), the time duration of the negative pulse is increased, whereby to minimize or substantially eliminate the stimulus artifact. When the stimulus artifact is outside the pre-defined limit and is rising upward (signifying that the tissue is over-discharged), the time duration of the negative pulse is decreased, whereby to minimize or substantially eliminate the stimulus artifact. The foregoing steps are repeated until the stimulus artifact is within the pre-defined limit, or until a time-out occurs (in which case the time-out will be reported to users). The previously-obtained optimum time duration of the negative pulse is then used as the initial time duration for the negative pulse of the next stimulation, and the foregoing steps are then repeated. Thus it will be seen that an automatic process can be used to determine the optimum time duration for the negative pulse of the biphasic stimulation so as to minimize or substantially eliminate stimulus artifact.

### (b) Using the Detected Voltage between the Anode and Cathode

The optimum time duration of the negative pulse can also be determined by recording, measuring and analyzing the voltage existing between anode 20 and cathode 15. This voltage is referred to as the Residual Voltage, or RV.

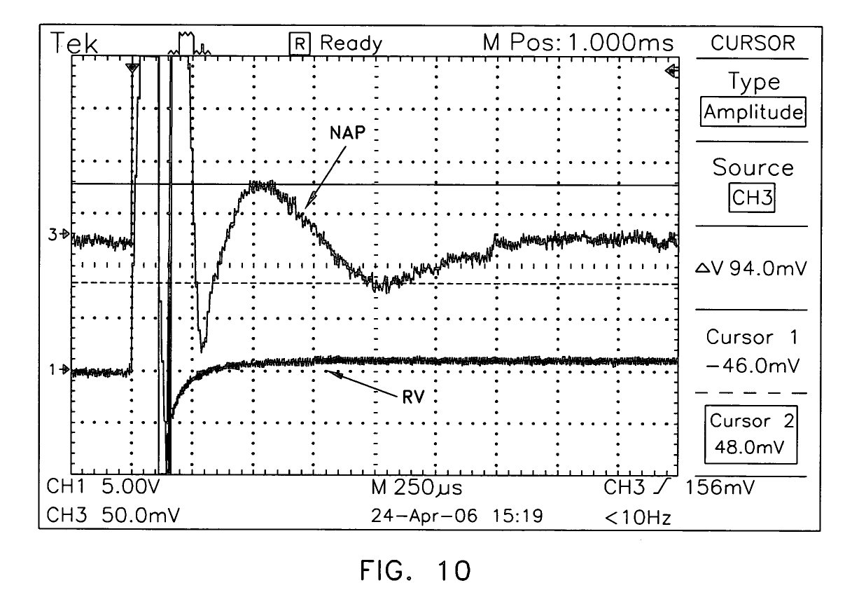

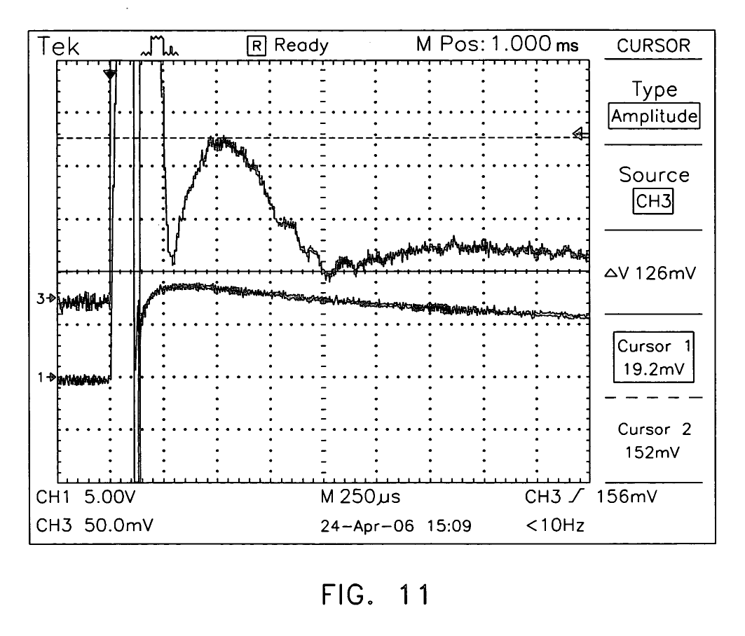

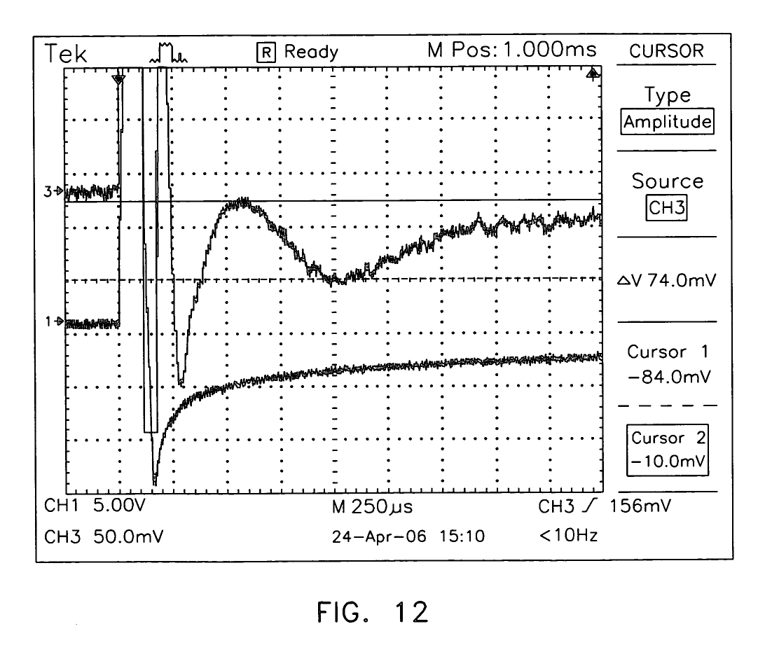

More particularly, and looking now at FIGS. 10-12, there are 2 traces on these figures, marked (on the left of each figure) as 1 and 3, corresponding to channel numbers 1 and 3 of the oscilloscope (channel numbers 2 and 4 were not used). Channel 1 is the voltage between anode 20 and cathode 15. Channel 3 is the trace signal detected by detector 40 with detection electrodes 25 and 30 connected. FIG. 10 illustrates that when the Residual Voltage (RV) during the NAP period (peak-to-peak) is low and flat, the stimulus artifact contamination is low, and the NAP measurement (94 mV) should be reliable (i.e., there is little or no stimulus artifact present in the trace signal shown by channel 3). FIG. 11 illustrates that when the RV during the NAP period (peak-to-peak) is falling down (signifying that the tissue is under-discharged), the channel 3 trace signal measurement (126 mV) is higher than the true NAP value (i.e., there is stimulus artifact present in the trace signal). FIG. 12 illustrates that when the RV during the NAP period (peak-to-peak) is rising up (signifying that the tissue is over-discharged), the channel 3 trace signal measurement (74 mV) is lower than the true NAP value (i.e., there is stimulus artifact present in the channel 3 trace signal).

In one preferred form of the present invention, the system is configured to compare the amplitude (or alternatively, the power) of the RV during the NAP period (peak-to-peak) to a pre-defined limit. When the RV is outside the pre-defined limit and is falling down, the time duration of the negative pulse is increased so as to reduce the stimulus artifact. When the RV is outside the pre-defined limit and is rising up, the time duration of the negative pulse is decreased. These steps are repeated until the RV is within the pre-defined limit, or until a time-out occurs (in which case the time-out will be reported to users). When the RV is within the pre-defined limit, the stimulus artifact contaminating the detected trace signal will have been eliminated (or at least reduced to an acceptable level) and the trace signal will provide a usable indication of the NAP signal.

### (ii) Automatic Stimulus Artifact Removal in Real-Time

A method for eliminating the stimulus artifact in real-time can also be implemented by detecting and measuring the voltage between anode 20 and cathode 15.

More particularly, if the impedance between anode 20 and cathode 15 was purely resistive, the voltage between the anode and cathode would drop to zero immediately after the end of the positive pulse. In this case, no negative pulse would need to be delivered.

On the other hand, if the impedance between anode 20 and cathode 15 was purely capacitive, the voltage between the anode and cathode would drop to zero after the same amount of current with opposite phase is delivered, i.e., after delivery of an appropriate negative pulse.

In humans, the impedance between anode 20 and cathode 15 has both resistive and capacitive components. More particularly, after delivery of the positive pulse, the voltage between the anode and cathode will drop to zero before the same amount of current with opposite phase (the negative pulse) is fully delivered, since the tissue is also self-discharging. When the amplitude of the negative pulse is the same as the amplitude of the positive pulse, the time duration of the negative pulse can be adjusted according to the time it takes for the voltage between the anode and cathode to drop to zero.

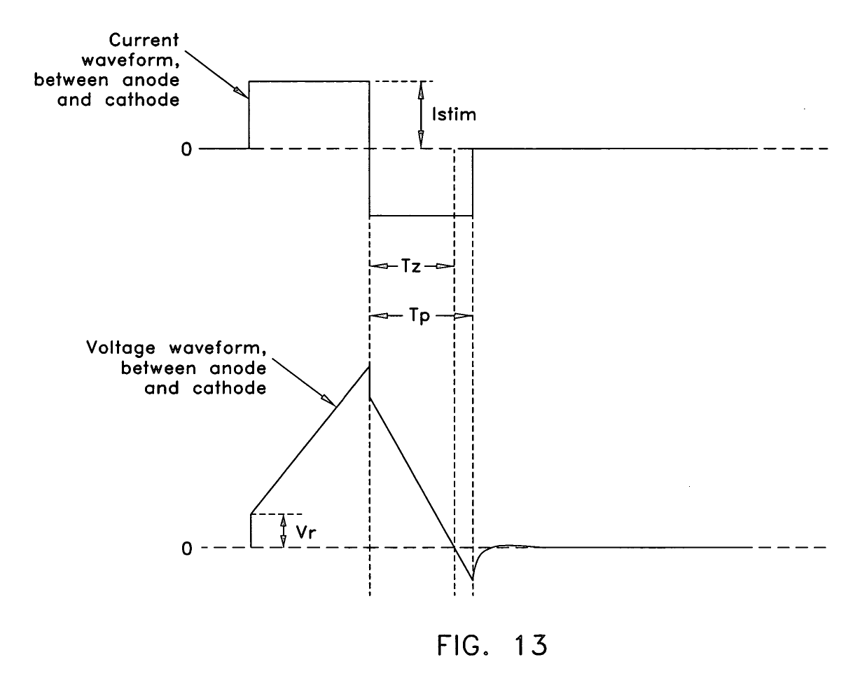

Looking now at FIG. 13, Tz reflects the amount of energy that is self-discharged (i.e., by the tissue discharge current) during the time that the negative pulse is being delivered. The greater the value of Tz, the slower the tissue's discharge speed, and therefore the longer the time duration needed for the negative pulse (Tp).

The impedance measurements between anode 20 and cathode 15 can also help to determine the time duration needed for the negative pulse to minimize the stimulus artifact. The present invention can measure serial capacitance, serial resistance, parallel capacitance, and parallel resistance between the anode and cathode. These parameters can be used to estimate the values of the capacitors C1 and C2, and the values of the resistors R1 and R2, in the simplified impedance model of FIG. 2. The device can also measure the value of R3+R4 in FIG. 2 by applying a small constant current to the anode and cathode. Refer to FIG. 13. R3+R4=Vr/Istim. The present invention measures the impedance parameters before stimulation, and then simulates the tissue discharge process using the appropriate impedance model, such as the one shown in FIG. 2, and determines the time duration which should be used for the negative pulse.

### Further Reflections on the Present Invention

It should be appreciated that the present invention is different from the prior art in all of the following aspects, among others:

(1) The present invention provides a determination of the relationship between the NAP amplitude and the conduction distance d from the stimulation cathode to the detecting electrodes.

(2) The present invention discloses a method for non-invasively acquiring a nerve action potential (NAP) of a patient as large as several hundred microvolts. This method involves placing detecting electrodes as close as 2 cm away from the stimulation site, and using a low pass filter that has high cut-off frequency. This method is useful for stimulation studies that use both surface electrodes and needle electrodes.

(3) The present invention discloses a method for using biphasic stimulation, wherein the negative pulse has constant current but adjusted time duration, so as to minimize or substantially eliminate stimulus artifacts.

(4) The present invention discloses a method for (i) detecting the voltage between stimulation anode and cathode, and (ii) using that detected voltage to determine the level of stimulus artifact contaminating the trace signal detected by detector 40.

(5) The present invention discloses methods for automatically reducing stimulus artifacts.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. Apparatus for acquiring a nerve action potential (NAP) from a patient, the apparatus comprising:
a stimulator and a pair of stimulator electrodes connected to the stimulator for applying an electrical stimulus to the patient so as to evoke a nerve action potential (NAP) in the patient;
a detector and a pair of detector electrodes connected to the detector for acquiring a trace signal from the patient, wherein the trace signal includes the nerve action potential (NAP); and
shorting apparatus for shorting the pair of stimulator electrodes after application of the electrical stimulus to the patient in order to minimize the presence of stimulus artifacts in the trace signal.

2. Apparatus according to claim 1 further including a reference electrode for providing an electrical reference with respect to the pair of detector electrodes, the reference electrode being connected to the detector.

3. Apparatus according to claim 1 wherein the shorting apparatus is formed internal to the stimulator.

4. Apparatus according to claim 1 wherein the electrical stimulus comprises a monophasic electrical stimulus.

5. Apparatus according to claim 1 wherein the electrical stimulus comprises a biphasic electrical stimulus comprising a positive pulse and a negative pulse.

6. Apparatus according to claim 5 wherein the shorting apparatus is configured to short the pair of stimulator electrodes after application of the positive pulse and before application of the negative pulse.

7. Apparatus according to claim 1 wherein the shorting apparatus is configured to short the pair of stimulator electrodes in order to minimize the presence of stimulus artifacts at least one of: (i) the time of the up-peak of the nerve action potential (NAP), and (ii) the time of the down-peak of the nerve action potential (NAP).

8. Apparatus according to claim 1 wherein the shorting apparatus is configured to short the pair of stimulator electrodes in order to minimize the presence of stimulus artifacts in the trace signal after the end of the electrical stimulus.

9. A method for acquiring a nerve action potential (NAP) from a patient, the method comprising the steps of:
applying an electrical stimulus to the patient using a stimulator and a pair of stimulator electrodes connected to the stimulator so as to evoke a nerve action potential (NAP) in the patient; and
acquiring a trace signal from the patient which includes the nerve action potential (NAP);
wherein the pair of stimulator electrodes are shorted after the electrical stimulus has been applied to the patient in order to minimize the presence of stimulus artifacts in the trace signal.

10. A method according to claim 9 wherein a reference electrode is used to acquire the trace signal from the patient.

11. A method according to claim 9 wherein the shorting of the pair of stimulator electrodes is effected by a component internal to the stimulator.

12. A method according to claim 9 wherein the electrical stimulus comprises a monophasic electrical stimulus.

13. A method according to claim 9 wherein the electrical stimulus comprises a biphasic electrical stimulus comprising a positive pulse followed by a negative pulse.

14. A method according to claim 13 wherein the shorting of the pair of stimulator electrodes is effected after application of the positive pulse and before application of the negative pulse.

15. A method according to claim 9 wherein the shorting of the pair of stimulator electrodes is effected so as to minimize the presence of stimulus artifacts at least one of: (i) the time of the up-peak of the nerve action potential (NAP), and (ii) the time of the down-peak of the nerve action potential (NAP).

16. A method according to claim 9 wherein the shorting of the pair of stimulator electrodes is effected so as to minimize the presence of stimulus artifacts in the trace signal after the end of the electrical stimulus.

17. Apparatus for acquiring a nerve action potential (NAP) from a patient, the apparatus comprising:
a stimulator and a pair of stimulator electrodes connected to the stimulator for applying an electrical stimulus to the patient so as to evoke a nerve action potential (NAP) in the patient;
a detector and a pair of detector electrodes connected to the detector for acquiring a trace signal from the patient, wherein the trace signal includes the nerve action potential (NAP);
wherein the stimulator is configured to produce a biphasic electrical stimulus consisting of a positive pulse followed by a negative pulse;
and further wherein the stimulator is configured to minimize the presence of stimulus artifacts in the trace signal by regulating the time duration of the negative pulse.

18. Apparatus according to claim 17 further including a reference electrode for providing an electrical reference with respect to the pair of detector electrodes, the reference electrode being connected to the detector.

19. Apparatus according to claim 17 wherein the stimulator is configured to regulate the time duration of the negative pulse in order to minimize the presence of stimulus artifacts at least one of: (i) the time of the up-peak of the nerve action potential (NAP), and (ii) the time of the down-peak of the nerve action potential (NAP).

20. Apparatus according to claim 17 wherein the stimulator is configured to regulate the time duration of the negative pulse in order to minimize the presence of in order to minimize the presence of stimulus artifacts in the trace signal after the end of the electrical stimulus.

21. Apparatus according to claim 17 further including shorting apparatus for shorting the pair of stimulator electrodes after application of the electrical stimulus to the patient in order to minimize the presence of stimulus artifacts in the trace signal.

22. Apparatus according to claim 21 wherein the shorting apparatus is configured to short the pair of stimulator electrodes after application of the positive pulse and before application of the negative pulse.

23. Apparatus according to claim 21 wherein the shorting apparatus is formed internal to the stimulator.

24. Apparatus according to claim 21 wherein the shorting apparatus is configured to short the pair of stimulator electrodes in order to minimize the presence of stimulus artifacts at least one of: (i) the time of the up-peak of the nerve action potential (NAP), and (ii) the time of the down-peak of the nerve action potential (NAP).

25. Apparatus according to claim 21 wherein the shorting apparatus is configured to short the pair of stimulator electrodes in order to minimize the presence of stimulus artifacts in the trace signal after the end of the electrical stimulus.

26. A method for acquiring a nerve action potential (NAP) from a patient, the method comprising the steps of:
applying an electrical stimulus to the patient so as to evoke a nerve action potential (NAP) in the patient, wherein the electrical stimulus comprises a biphasic electrical stimulus comprising a positive pulse followed by a negative pulse; and
acquiring a trace signal from the patient which includes the nerve action potential (NAP);
wherein the time duration of the negative pulse is regulated so as to minimize the presence of stimulus artifacts in the trace signal.

27. A method according to claim 26 wherein the time duration of the negative pulse is regulated in order to minimize the presence of stimulus artifacts at least one of: (i) the time of the up-peak of the nerve action potential (NAP), and (ii) the time of the down-peak of the nerve action potential (NAP).

28. A method according to claim 26 wherein the time duration of the negative pulse is regulated in order to minimize the presence of stimulus artifacts in the trace signal after the end of the electrical stimulus.

29. A method according to claim 26 wherein the electrical stimulus is applied to the patient using a stimulator and a pair of stimulator electrodes connected to the stimulator, and further wherein the pair of stimulator electrodes are shorted after the electrical stimulus has been applied to the patient in order to minimize the presence of stimulus artifacts in the trace signal.

30. A method according to claim 29 wherein the pair of stimulator electrodes are shorted after application of the positive pulse and before application of the negative pulse.

31. A method according to claim 29 wherein the shorting of the pair of stimulator electrodes is effected by a component internal to the stimulator.

32. A method according to claim 29 wherein the shorting of the pair of stimulator electrodes is effected so as to minimize the presence of stimulus artifacts at least one of: (i) the time of the up-peak of the nerve action potential (NAP), and (ii) the time of the down-peak of the nerve action potential (NAP).

33. A method according to claim 29 wherein the shorting of the pair of stimulator electrodes is effected so as to minimize the presence of stimulus artifacts in the trace signal after the end of the electrical stimulus.

34. Apparatus for acquiring a nerve action potential (NAP) from a patient, the apparatus comprising:
a stimulator and a pair of stimulator electrodes connected to the stimulator for applying an electrical stimulus to the patient so as to evoke a nerve action potential (NAP) in the patient, wherein the stimulator is configured to produce a biphasic electrical stimulus consisting of a positive pulse followed by a negative pulse;
a detector and a pair of detector electrodes connected to the detector for acquiring a trace signal from the patient, wherein the trace signal includes the nerve action potential; and
a determining component for determining the amplitude of a stimulus artifact present in the trace signal;
wherein the stimulator is configured to minimize the presence of stimulus artifacts in the trace signal by regulating the time duration of the negative pulse based on the amplitude of a stimulus artifact present in a prior trace signal as determined by the determining component.

35. Apparatus according to claim 34 wherein the stimulator is configured to regulate the time duration of the negative pulse in order to minimize the presence of stimulus artifacts at least one of: (i) the time of the up-peak of the nerve action potential (NAP), and (ii) the time of the down-peak of the nerve action potential (NAP).

36. Apparatus according to claim 34 wherein the stimulator is configured to regulate the time duration of the negative pulse in order to minimize the presence of stimulus artifacts in the trace signal after the end of the electrical stimulus.

37. Apparatus according to claim 34 wherein the determining component utilizes the trace signal and a feedback mechanism applied across multiple applications of the electrical stimulus in order to determine the amplitude of the stimulus artifact.

38. Apparatus according to claim 34 wherein the determining component detects the voltage between the pair of stimulator electrodes during the biphasic electrical stimulus, during the application of the positive pulse and during the application of the negative pulse, in order to determine the amplitude of the stimulus artifact.

39. Apparatus according to claim 34 wherein the determining component uses a tissue impedance model and tissue impedance measurements in order to predict the amplitude of the stimulus artifact in real-time.

40. Apparatus according to claim 39 wherein the tissue measurements comprise serial capacitance and serial resistance; and/or parallel capacitance and parallel resistance between the pair of stimulator electrodes.

41. Apparatus according to claim 34 further including a reference electrode for providing an electrical reference with respect to the pair of detector electrodes, the reference electrode being connected to the detector.

42. Apparatus according to claim 34 further including shorting apparatus for shorting the pair of stimulator electrodes after application of the electrical stimulus to the patient in order to minimize the presence of stimulus artifacts in the trace signal.

43. Apparatus according to claim 42 wherein the shorting apparatus is configured to short the pair of stimulator electrodes after application of the positive pulse and before application of the negative pulse.

44. Apparatus according to claim 42 wherein the shorting apparatus is formed internal to the stimulator.

45. Apparatus according to claim 42 wherein the shorting apparatus is configured to short the pair of stimulator electrodes in order to minimize the presence of stimulus artifacts at least one of: (i) the time of the up-peak of the nerve action potential (NAP), and (ii) the time of the down-peak of the nerve action potential (NAP).

46. Apparatus according to claim 42 wherein the shorting apparatus is configured to short the pair of stimulator electrodes in order to minimize the presence of stimulus artifacts in the trace signal after the end of the electrical stimulus.

47. A method for acquiring a nerve action potential (NAP) from a patient, the method comprising the steps of:
applying an electrical stimulus to the patient so as to evoke a nerve action potential (NAP) in the patient, wherein the electrical stimulus comprises a biphasic electrical stimulus comprising of a positive pulse followed by a negative pulse;
acquiring a trace signal from the patient which includes the nerve action potential (NAP);
determining the amplitude of a stimulus artifact present in the trace signal; and
regulating the time duration of the negative pulse in a subsequent biphasic electrical stimulus so as to minimize the presence of stimulus artifacts in a current trace signal based on the amplitude of a stimulus artifact present in a prior trace signal.

48. A method according to claim 47 wherein determining the amplitude of a stimulus artifact present in the trace signal is effected by utilizing the trace signal and a feedback mechanism applied across multiple applications of the electrical stimulus in order to determine the amplitude of the stimulus artifact.

49. A method according to claim 47 wherein the electrical stimulus is applied to the patient with a pair of stimulator electrodes, and further wherein determining the amplitude of a stimulus artifact present in the trace signal is effected by detecting the voltage between the pair of stimulator electrodes after the application of the positive pulse and before the application of the negative pulse in order to determine the amplitude of the stimulus artifact.

50. A method according to claim 47 wherein determining the amplitude of a stimulus artifact present in the trace signal is effected by using a tissue impedance model and tissue measurements in order to predict the amplitude of the stimulus artifact in real-time.

51. A method according to claim 47 wherein the tissue measurements comprise serial capacitance, serial resistance, parallel capacitance, and parallel resistance between the pair of stimulator electrodes.

52. A method according to claim 47 wherein the time duration of the negative pulse is regulated in order to minimize the presence of stimulus artifacts at least one of: (i) the time of the up-peak of the nerve action potential (NAP), and (ii) the time of the down-peak of the nerve action potential (NAP).

53. A method according to claim 47 wherein the time duration of the negative pulse is regulated in order to minimize the presence of in order to minimize the presence of stimulus artifacts in the trace signal after the end of stimulus.

54. Apparatus for measuring the stimulus artifact present when acquiring a nerve action potential (NAP) from a patient, the apparatus comprising:
a stimulator and a pair of stimulator electrodes connected to the stimulator for applying an electrical stimulus to the patient so as to evoke a nerve action potential (NAP) in the patient;
a detector and a pair of detector electrodes connected to the detector for acquiring a trace signal from the patient, wherein the trace signal includes the nerve action potential (NAP); and
a measuring component for measuring the voltage present between the pair of stimulator electrodes after application of the electrical stimulus to the patient.

55. Apparatus according to claim 54 wherein the electrical stimulus comprises a biphasic electrical stimulus comprising a positive pulse followed by a negative pulse, and further wherein the measuring component measures the voltage present between the pair of stimulator electrodes after application of the positive pulse and before the complete application of the negative pulse.

56. Apparatus according to claim 55 wherein the measuring component measures the voltage present between the pair of stimulator electrodes at least one of: (i) the time of the up-peak of the nerve action potential (NAP), and (ii) the time of the down-peak of the nerve action potential (NAP).

57. A method for measuring the stimulus artifact present when acquiring a nerve action potential (NAP) from a patient, the method comprising the steps of:
applying an electrical stimulus to the patient using a pair of stimulator electrodes, so as to evoke a nerve action potential (NAP) in the patient; and
acquiring a trace signal from the patient which includes the nerve action potential (NAP);
wherein the voltage between the pair of stimulator electrodes is measured after the beginning of application of the electrical stimulus to the patient.

58. A method according to claim 57 wherein the electrical stimulus comprises a biphasic electrical stimulus comprising a positive pulse followed by a negative pulse, and further wherein the voltage between the pair of stimulator electrodes is measured during application of the positive pulse and during the application of the negative pulse.

59. Apparatus according to claim 58 wherein the voltage between the pair of stimulator electrodes is measured at least one of: (i) the time of the up-peak of the nerve action potential (NAP), and (ii) the time of the down-peak of the nerve action potential (NAP).

60. Apparatus for acquiring a large nerve action potential (NAP) from a patient, the apparatus comprising:
a stimulator and a pair of stimulator electrodes connected to the stimulator for applying an electrical stimulus to the patient so as to evoke a nerve action potential (NAP) in the patient; and
a detector and a pair of detector electrodes connected to the detector for acquiring a trace signal from a patient, wherein the trace signal includes the nerve action potential (NAP);
wherein at least one of the pair of detector electrodes is a surface electrode and is positioned less than 3 cm from the stimulator electrodes.

61. A method for acquiring large nerve action potential (NAP) from a patient, the method comprising the steps of:
applying an electrical stimulus to the patient using a pair of stimulator electrodes so as to evoke a nerve action potential (NAP) in the patient; and
acquiring a trace signal from the patient which includes the nerve action potential (NAP), wherein the trace signal is acquired from the patient using a pair of detector electrodes;
wherein at least one of the pair of the detector electrodes is a surface electrode and is placed less than 3 cm from the stimulator electrodes.

62. Apparatus according to claim 1 wherein the apparatus further comprises a controller/monitor for recording, measuring and analyzing the trace signal acquired by the detector and the pair of detector electrodes, wherein the controller/monitor is connected to the detector via a wireless connection.

63. A method according to claim 9 wherein the trace signal acquired from the patient is sent via a wireless connection to a controller/monitor for recording, measuring and analyzing.

64. Apparatus according to claim 17 wherein the apparatus further comprises a controller/monitor for recording, measuring and analyzing the trace signal acquired by the detector and the pair of detector electrodes, wherein the controller/monitor is connected to the detector via a wireless connection.

65. A method according to claim 26 wherein the trace signal acquired from the patient is sent via a wireless connection to a controller/monitor for recording, measuring and analyzing.

66. Apparatus according to claim 34 wherein the apparatus further comprises a controller/monitor for recording, measuring and analyzing the trace signal acquired by the detector and the pair of detector electrodes, wherein the controller/monitor is connected to the detector via a wireless connection.

67. A method according to claim 47 wherein the trace signal acquired from the patient is sent via a wireless connection to a controller/monitor for recording, measuring and analyzing.

68. Apparatus according to claim 54 wherein the apparatus further comprises a controller/monitor for recording, measuring and analyzing the trace signal acquired by the detector and the pair of detector electrodes, wherein the controller/monitor is connected to the detector via a wireless connection.

69. A method according to claim 57 wherein the trace signal acquired from the patient is sent via a wireless connection to a controller/monitor for recording, measuring and analyzing.

70. Apparatus according to claim 60 wherein the apparatus further comprises a controller/monitor for recording, measuring and analyzing the trace signal acquired by the detector and the pair of detector electrodes, wherein the controller/monitor is connected to the detector via a wireless connection.

71. A method according to claim 61 wherein the trace signal acquired from the patient is sent via a wireless connection to a controller/monitor for recording, measuring and analyzing.
